(19) United States
(12) Patent Application Publication
(10) Pub. No.: US 2002/0098247 A1
(43) Pub. Date: Jul. 25, 2002

(54) Methods and compositions for the improvement of insulin sensitivity, reduction of hyperglycemia, and reduction of hypercholesterolemia with chromium complexes and alpha lipoic acid

(76) Inventors: James R. Komorowski, Trumbull, CT (US); Danielle Greenberg, Waccabuc, NY (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/001,322

(22) Filed: Oct. 31, 2001

Related U.S. Application Data
(60) Provisional application No. 60/245,705, filed on Nov. 2, 2000.

(51) Int. Cl. A61K 33/24; A61K 35/78

(52) U.S. Cl. 424/655; 424/725

(57) ABSTRACT

A composition for improving insulin sensitivity, reducing hyperglycemia, and reducing hypercholesterolemia, the composition including at least one chromium complex and alpha-lipoic acid is disclosed. A method of improving insulin sensitivity in a subject in need thereof including administering to a subject a pharmaceutically effective dose of alpha-lipoic acid in conjunction with at least one chromium complex selected from the group consisting of chromium picolinate, chromium nicotinate, chromic tripicolinate, chromic polynicotinate, chromium chloride, chromium histidinate, and chromium yeasts is also provided.

### RELATED APPLICATION

This application claims priority to Provisional Application No. 60/245,705 entitled METHODS AND COMPOSITIONS FOR THE IMPROVEMENT OF INSULIN SENSITIVITY, REDUCTION OF HYPERGLYCEMIA, AND REDUCTION OF HYPERCHOLESTEROLEMIA filed on Nov. 2, 2000. The subject matter of the aforementioned application is hereby incorporated by reference.

### BACKGROUND OF THE INVENTION

1. Field of the Invention

The disclosed invention relates to compositions and methods for the improvement of insulin sensitivity, reduction of hyperglycemia, and reduction of hypercholesterolemia. Specifically, compositions comprising chromium complexes in combination with alpha-lipoic acid are provided.

2. Description of the Related Art

Diabetes mellitus is a chronic disorder characterized by impaired metabolism of glucose and other energy-yielding fuels, and the late development of vascular and neuropathic complications. In this group of disorders with distinct pathogenic mechanisms, hyperglycemia is the common denominator. Independent of the cause, the disease is associated with insulin deficiency, which may be total, partial, or relative when viewed in the context of coexisting insulin resistance.

Experimentally induced diabetes in animals produces many of the same microvascular and neuropathologic changes observed in human diabetics. Treatment with alpha-lipoic acid has been shown to prevent many of the neuropathologic and microvascular changes in animals (Nagamatsu M, et al., Diabetes Care 18:1160, 1995). Alpha-lipoic acid (also called lipoic acid or thioctic acid) is a sulfur-containing lipid that is readily converted to and from its reduced form, dihydrolipoic acid. It acts as a coenzyme in reactions that occur in the Krebs cycle; specifically it is involved in the decarboxylation of pyruvate and some other alpha-keto acids.

Some parameters that are typically abnormal in diabetes, and which are corrected by alpha-lipoic acid treatment, include digital nerve conduction (DNC) velocity, neural blood flow and glutathione levels. Interestingly, the levels of the important antioxidant glutathione are reduced in diabetes, and while exogenous administration of glutathione does not alter the levels of glutathione, alpha-lipoic acid treatment does correct this abnormality, presumably by participating in antioxidant reactions both inside an outside the cell. In recent study in diabetic rats, alpha-lipoic acid treatment was found to prevent the deficit in acetylcholine-induced relaxation in isolated vasculature by 94 percent (Keegan A, et al., Diabetologia 42:343, 1999). Alpha-lipoic acid has also been shown to inhibit the production of glucose from protein sources in diabetic rats, thereby preserving protein stores and keeping additional unwanted glucose from the circulation (Khamaisi M, et al., Metabolism 48:504, 1999).

A number of other studies support the beneficial effects of alpha-lipoic acid treatment in experimental models of diabetic long-term complications with respect to glucose metabolic homeostasis in both nervous (Kishi Y, et al., Diabetes 48:2045, 1999) and ocular tissues (Obrosova I, et al., Diabetologia 41:1442, 1998).

The administration of alpha-lipoic acid to treat diabetes has also been investigated in human subjects. Following its introduction in Germany in 1959 for the treatment of liver failure associated with the ingestion of toxic mushrooms (Amantia phalloides), alpha-lipoic acid was tested in diabetic patients with neuropathic complaints. While at that time the exact mechanism(s) by which alpha-lipoic acid was believed to act was not known, some had speculated that this agent enhanced glucose uptake into peripheral nerves. Additionally, others suggested that alpha-lipoic acid levels were depleted in diabetics and that the neuropathies noted in diabetes resulted from this deficit.

There were ten clinical studies performed between 1959 and 1993 to determine the utility of alpha-lipoic acid in diabetic polyneuropathy. (Ziegler D, Exp Clin Endocrinol Diabetes 107:421, 1999). While some of these studies demonstrated beneficial results on symptoms and nerve function tests, most had design flaws that prevent an unequivocal conclusion regarding the effectiveness of this treatment. Typically, doses of alpha-lipoic acid of 100-600 mg per day for durations ranging from a few weeks to a number of months yielded some promising results. Of the 10 studies, only three were double-blinded, and together these constituted only 76 patients. One study showed a clear positive effect, another a trend, and the third no significant effects. While the remaining studies, which were either single-blinded or open-label, demonstrated significant beneficial effects in all but one study, the significance of such nondouble-blinded studies is considered less useful by many investigators.

The first controlled clinical trial of alpha-lipoic acid was conducted by Ziegler and co-workers in 1995 and was called the Alpha-Lipoic Acid in Diabetic Neuropathy (ALADIN) Study (Ziegler D, et al., Diabetologia 38:1425, 1995). Patients (N=328) with Type II diabetes who suffered from symptomatic polyneuropathy, received intravenous placebo or one of three doses of alpha-lipoic acid (100, 600, 1,200 mg per day) for three weeks. The Total Symptom Score (TSS), which measures in intensity and frequency of pain, parasthesias, burning and numbness, decreased significantly by approximately 60 percent in the feet with two higher doses of alpha-lipoic acid. These beneficial effects were noted as early as five days after the initiation of therapy, and the frequency of adverse effects was similar among all the groups with the exception of the 1,200 mg dose group, which experienced a slightly greater rate of gastrointestinal side effects.

The next follow-up investigation, the ALADIN II Study, utilized a combination of intravenous dosing and oral dosing of placebo and alpha-lipoic acid at levels of 600 or 1,200 mg per day for two years (Reljanovic M, et al., Free Radical Res 31:171, 1999). Intravenous administration for five days was followed by oral administration for the next two years. Sixty-five patients with Type I and Type II diabetes completed the study, which demonstrated improvements in sensory nerve and tibial nerve conduction velocities with both doses of alpha-lipoic acid. In this study, the Neuropathy Disability Score values were not significantly affected by either dose of alpha-lipoic acid. There were no significant differences between placebo and alpha-lipoic acid group with respect to the incidence or the severity of side effects during this two-year study.

The ALADIN III study utilized placebo and alpha-lipoic acid (600 mg) administered intravenously for three weeks followed by 6 months of three times a day (tid) oral treatments to 509 patients with Type II diabetes (Ziegler D, et al., Diabetes Care 22:1296, 1999). As evidenced by the TSS, neuropathy was significantly lessened in the alpha-lipoic acid group as compared with the placebo group. These beneficial effects were noted as early as three weeks into the study, and again, there were no significant differences between group with respect to side effects.

The Oral Pilot (OPRIL) Study aimed to determine the effectiveness of alpha-lipoic acid administered orally (Ruhnau K J, et al., Diabet Med 16:1040, 1999). In the 24 patients with Type II diabetes receiving either placebo or alpha-lipoic acid, 600 mg tid, for three weeks, improvements in TSS in the feet and NDS scores were noted. The plasma levels obtained with 600 mg tid orally had been previously shown to be similar to those obtained following 600 mg intravenously. No significant differences in side effects were noted. A subsequent study that demonstrated improvements in cardiac autonomic dysfunction using 800 mg per day orally for four months similarly demonstrated the relatively safe use of alpha-lipoic acid.

A long-term (four year) study is being carried out to determine the effectiveness of orally administered alpha-lipoic acid with respect to the neuropathies associated with diabetes mellitus. In this Neurological Assessment of Thioctic Acid in Neuropathy (NARHAN) I Study, results should become available in the next few years. Another similar study, NARHAN II, is designed to test parentally administered alpha-lipoic acid.

Dietary supplementation of chromium to normal individuals has been reported to lead to improvements in glucose tolerance, serum lipid concentrations, including high-density lipoprotein cholesterol, insulin and insulin binding (Anderson, Clin. Psychol. Biochem. 4:31-41, 1986). Supplemental chromium in the trivalent form, e.g. chromic chloride, is associated with improvements of risk factors associated with adult-onset (Type II) diabetes and cardiovascular disease.

Chromium is a nutritionally essential trace element. The essentiality of chromium in the diet was established in 1959 by Schwartz, as cited in Present Knowledge in Nutrition, page 571, fifth edition (1984, the Nutrition Foundation, Washington, D.C.). Chromium depletion is characterized by the disturbance of glucose, lipid and protein metabolism and by a shortened lifespan. Chromium is essential for optimal insulin activity in all known insulin-dependent systems (Boyle et al., Southern Med. J. 70:1449-1453, 1977). Insufficient dietary chromium has been linked to both maturity-onset diabetes and to cardiovascular disease.

The principal energy sources for the body are glucose and fatty acids. Chromium depletion results in biologically ineffective insulin and compromised glucose metabolism. Under these conditions, the body must rely primarily on lipid metabolism to meet its energy requirements, resulting in the production of excessive amounts of acetyl-CoA and ketone bodies. Some of the documented acetyl-CoA is diverted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia (Boyle et al., supra.).

Chromium functions as a cofactor for insulin. It binds to the insulin receptor and potentiates many, and perhaps all, of its functions (Boyle et al., supra.). These functions include, but are not limited to, the regulation of carbohydrate and lipid metabolism. (Present Knowledge in Nutrition, supra, at p. 573-577). The introduction of inorganic chromium compounds per se into individuals is not particularly beneficial. Chromium must be converted endogenously into an organic complex or must be consumed as a biologically active molecule. Only about 0.5% of ingested inorganic chromium is assimilated into the body (Recommended Daily Allowances, Ninth Revised Edition, The National Academy of Sciences, page 160, 1980). Only 1-2% of most organic chromium compounds are assimilated into the body.

U.S. Pat. Re. No. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible and easy to produce. These exogenously synthesized essential metal coordination complexes of picolinic acid (pyridine-2-carboxylic acid) have the following structural formula:

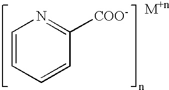

wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, the entire contents of which are hereby incorporated by reference, describes the administration of chromic tripicolinate for the treatment of adult-onset diabetes in doses ranging from 50 to 500 μg. International Patent Application No. WO96/35421 discloses the use of high doses of chromic tripicolinate (providing 1,000-10,000 μg chromium/day) for reducing hyperglycemia and stabilizing the level of serum glucose in humans with Type II diabetes. U.S. Pat. No. 5,789,401 discloses a chromic tripicolinate-biotin composition and its use in lowering blood glucose levels in humans with Type II diabetes.

U.S. Pat. Nos. 5,087,623; 5,087,624; and 5,175,156, the entire contents of which are hereby incorporated by reference, disclose the use of chromium tripicolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum HDL-cholesterol levels. U.S. Pat. Nos. 4,954,492 and 5,194,615, the entire contents of which are hereby incorporated by reference, describe a related complex, chromic nicotinate, which is also used for supplementing dietary chromium and lowering serum lipid levels. Picolinic acid and nicotinic acid are position isomers having the following structures:

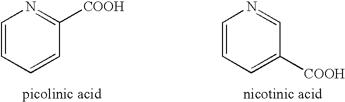

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin (Davis et al., J. Nutrition Res. 15:202-210, 1995; Kamath et al., J. Nutrition 127:478-482, 1997). These drugs inhibit the enzyme cyclooxygenase which converts arachidonic acid to various prostaglandins, resulting in inhibition of intestinal mucus formation and lowering of intestinal pH which facilitates chromium absorption.

Additional pharmacological compositions and methods for improving insulin sensitivity, reducing hyperglycemia, and reducing hypercholesterolemia are needed. A new, more effective, less expensive treatment for obesity with minimal side effects would be a boon to reducing the symptoms associated with diabetes.

### SUMMARY OF THE INVENTION

The present invention is directed to compositions and methods for improving insulin sensitivity, reducing hyperglycemia, and reducing hypercholesterolemia. In one aspect of the invention, a composition including at least one chromium complex and alpha-lipoic acid is disclosed. Advantageously, the chromium complex may include chromium picolinate, chromium nicotinate, chromic tripicolinate, chromic polynicotinate, chromium chloride, chromium histidinate, or chromium yeasts.

In one aspect of the invention, the composition may include a chelating agent. The chelating agent may be picolinic acid, nicotinic acid, or both.

In another aspect of the invention, the composition is incorporated into a pharmaceutically effective carrier. The pharmaceutically effective carrier can be a tablet, capsule, microbead, emulsion, powder, granule, suspension, syrup and elixir. Advantageously, the microbead is a sugar beadlet or microcrystalline cellulose beadlet. Preferably, the chromium complex and alpha-lipoic acid are coated on the beadlet. Optionally, the tablet, capsule, or microbead is coated with an enteric coating.

In yet another aspect of the invention, the chromium complex and the alpha-lipoic acid are in a ratio of between about 1:25 to 1:1000 (w/w).

Advantageously, the composition may include at least one of a cyclooxygenase inhibitor, a mucolytic, and a salicin-containing herb. The cyclooxygenase inhibitor may include indomethacin, ibuprofen, acetaminophen, or naproxen. The salicin-containing herb may include Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (balm of Gilead) or Salix alba (white willow). Preferably, the mucolytic is guaifenesin.

A method of improving insulin sensitivity in a subject in need thereof is likewise contemplated. The method includes administering to a subject a pharmaceutically effective dose of alpha-lipoic acid in conjunction with at least one chromium complex. The chromium complex can be chromium picolinate, chromium nicotinate, chromic tripicolinate, chromic polynicotinate, chromium chloride, chromium histidinate, or chromium yeasts.

The method may include the administration of at least one uncomplexed chelating agent such as picolinic acid, nicotinic acid, or both. Advantageously, the method further includes the administration of at least one of a cyclooxygenase inhibitor, a mucolytic, and a salicin-containing herb. Preferably, the cyclooxygenase inhibitor is indomethacin, ibuprofen, acetaminophen, or naproxen. In yet another aspect of the invention, the salicin-containing herb may include Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (balm of Gilead) or Salix alba (white willow). Advantageously, the mucolytic is guaifenesin.

A method of reducing hyperglycemia in a subject in need thereof is provided. A subject is administered a pharmaceutically effective dose of alpha-lipoic acid in conjunction with at least one chromium complex. Preferably, the chromium complex is chromium picolinate, chromium nicotinate, chromic tripicolinate, chromic polynicotinate, chromium chloride, chromium histidinate, or chromium yeasts. In some embodiments, a chelating agent such as picolinic acid, nicotinic acid, or both picolinic acid and nicotinic acid are administered.

In yet another aspect of the invention, a method of reducing hypercholesterolemia in a subject in need thereof is disclosed. The method includes administering a pharmaceutically effective dose of alpha-lipoic acid in conjunction with at least one chromium complex to a subject. The chromium complex may include chromium picolinate, chromium nicotinate, chromic tripicolinate, chromic polynicotinate, chromium chloride, chromium histidinate, or chromium yeasts. Optionally, the method of reducing hypercholesterolemia additionally includes administering a chelating agent such as picolinic acid, nicotinic acid, or both picolinic acid and nicotinic acid.

### DETAILED DESCRIPTION OF THE PREFERRED EMBODIMENT

The disclosed invention relates to compositions for the reduction of symptoms associated with diabetes. Additionally, methods for improving insulin sensitivity, reducing hyperglycemia, and reducing hypercholesterolemia are likewise contemplated. A primary basis of the present invention is the novel and unexpected discovery that the coadministration of an effective dose of a chromium complex in combination with alpha-lipoic acid produces a synergistic improvement of insulin sensitivity, reduction of hyperglycemia, and reduction of hypercholesterolemia.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described.

Insulin resistance of skeletal muscle glucose uptake is a prominent feature of Type II diabetes and pharmacological interventions are directed to improving insulin sensitivity. Complexes of chromium have also been shown to improve insulin sensitivity in a number of experimental and clinical trials. As used herein, the term “chromium complexes” or “chromium complex” includes, without limitation, chromium picolinate, chromic tripicolinate, chromium nicotinate, chromic polynicotinate, chromium chloride, chromium histidinate, and chromium yeasts. Over a dozen studies in the last 30 years have reported on the effects of chromium supplementation in subjects with Type II diabetes. Chromium picolinate, for example, has been reported to generate positive effects in terms of increasing insulin sensitivity. There have been no documented toxic effects in any of the human studies involving supplemental chromium, on people with varying degrees of glucose intolerance ranging from mild glucose intolerance to overt Type II diabetes.

There have been more than 23 published chromium supplementation studies over the past three decades involving subjects who do not have clinical diabetes. All but five of these reported at least one significant positive effect of supplemental chromium. The most readily observed benefit reported in the majority of the studies was improved blood sugar and/or insulin.

In addition to improvements in blood glucose and insulin due to supplemental chromium complexes, there have been at least eight studies involving chromium supplementation of subjects without diabetes whose blood lipids improved following chromium supplementation. Such improvements are usually greatest in subjects with the highest blood lipids, but significant changes may take several months to appear. In some embodiments, compositions comprising an effective dose of a chromium are provided. As used herein, an effective dose of a chromium complex is preferably a dose that would provide between about 100 μg/day to about 2000 μg/day of chromium. Specifically, the effective dose of chromium is about 200 μg/day, 400 μg/day, 500 μg/day, 600 μg/day, 800 μg, 1,000 μg/day, or 1,500 μg/day. Preferably, the effective dose of chromium is 1,000 μg/day.

The compositions additionally include an effective dose of alpha-lipoic acid (ALA). Alpha-lipoic acid (ALA), a natural occurring compound frequently used for treatment of diabetic polyneuropathy, enhances glucose utilization in various experimental models. Advantageously, the composition will include alpha-lipoic acid at a dose of between about 10 to 1,000 mg/day, more preferably, the dosage is between about 50 to 500 mg/day. Most preferably, the dosage is about 100 mg/day.

Advantageously, the compositions include chromium complex and alpha-lipoic acid in a ratio of between about 1:5 to 1:10,000 (w/w). Preferably, the ratio of chromium complex to alpha-lipoic acid is between about 1:25 to 1:1000 (w/w). Most preferably, the compositions comprise chromium complex and alpha-lipoic acid in a ratio of between about 1:100 (w/w).

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include picolinic acid, nicotinic acid, or both picolinic acid and nicotinic acid. Thus, the compositions of the disclosed invention are readily absorbable forms of chromium which also facilitate absorption of other essential metals in the human diet.

The chromium complexes of the disclosed invention have the same uses as described for chromic tripicolinate in U.S. Pat. Nos. 5,087,623, 5,087,624 and 5,174,156, namely supplementing dietary chromium, lowering blood glucose levels in diabetics, lowering serum lipid levels and increasing lean body mass. Additionally, the chromium complexes of the present invention act to treat symptoms associated with diabetes.

Advantageously, the chromium complexes are synthetic. The synthesis and use of chromium picolinates is described in U.S. Pat. Re. No. 33,988 and U.S. Pat. No. 5,087,623. Chromic tripicolinate is available from health food stores, drug stores and other commercial sources. The synthesis and use of chromic polynicotinate is described in U.S. Pat. No. 5,194,615.

The chelating agents such as picolinic acid and nicotinic acid are available from many commercial sources, including Sigma-Aldrich (St. Louis, Mo.) (picolinic acid; catalog No. P5503; nicotinic acid; catalog No. PN4126). Preferably, the ratio of the chromium complex to the chelating agent from about 10:1 to about 1:10 (w/w), more preferably from about 5:1 to about 1:5 (w/w). Alternatively, the molar ratio of chromium complex to the uncomplexed chelating agent is preferably 1:1, and may be from about 5:1 to about 1:10.

A variety of delivery systems are available to deliver the compositions to a subject in need thereof. Preferably, the compositions of the disclosed invention are prepared by incorporating the components into a pharmaceutically acceptable carrier, including but not limited to tablets, capsules and microbeads, preferably sugar beadlets or microcrystalline cellulose.

For oral administration, the chromium complex may be incorporated into a tablet, aqueous or oil suspension, dispersible powder or granule, microbead, emulsion, hard or soft capsule, syrup or elixir. The components of the composition may also be administered separately. Compositions may be prepared according to any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

In another preferred embodiment, tablets, capsules or microbeads are coated with an enteric coating which prevents dissolution in the acidic environment of the stomach. Instead, this coating dissolves in the small intestine at a more neutral pH. Because certain chromium complexes may be more stable at this neutral pH than at the acidic pH of the stomach, enhanced absorption occurs because the chromium complexes remain substantially intact until they reach the small intestine. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, D.C., 1998, the entire contents of which are hereby incorporated by reference.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the chromium complexes of the invention in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agent, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The oral formulations described above may also include aspirin (acetylsalicylic acid), other salicylates, or another NSAIDs such as indomethacin, ibuprofen, acetaminophen, naproxen or any drug capable of inhibiting the cyclooxygenase pathway leading to prostaglandin synthesis. This results in a decrease in intestinal mucus production and lower intestinal pH which facilitates absorption of the chromium compositions of the present invention. The oral compositions may further include mucolytics such as guaifenesin and the like, to inhibit intestinal mucus production, and/or acids such as ascorbic acid, citric acid and the like to lower intestinal pH. Inclusion of one or both of these compounds further enhances chromium absorption. There are two forms of cyclooxygenase (cox), cox1 and cox2, which differ in their sensitivity to inhibition by NSAIDs. The cox2 isozyme promotes prostaglandin formation at sites of inflammation, but not at other sites such as the gastrointestinal tract. In contrast, relatively selective inhibition of cox1 facilitates chromic tripicolinate and chromic polynicotinate absorption. Although the selective inhibition of cox1 is desirable, any inhibitor or cox1 or cox2 can be formulated with the chromic tripicolinate and chromic polynicotinate compositions of the invention. Cox inhibitors, acids and mucolytics may also be coadministered with the chromic tripicolinate and chromic polynicotinate compositions of the invention. The amount of these drugs formulated with or coadministered with the chromic tripicolinate compositions of the invention are as follows: cox inhibitions, between about 50 mg and 500 mg; mucolytics, between about 10 mg and 250 mg; and acids, between about 50 mg and about 1,000 mg.

The coadministration or formulation of salicin-containing herbs with the compositions of the invention is also contemplated. Class I herbs, as documented in the American Herbal Products Association's Botanical Safety Handbook (herbs that can be safely consumed when used appropriately), such as Boswelia serrata (frankincense), Betula lenta (sweet birch), Betula pubescens (white birch), Filipendula ulmaria (meadowsweet), Gaultheria procumbens (wintergreens), Populus balsamifera and Populus jackii (balm of Gilead), and Salix alba (white willow) are all salicin-containing plants with salicylate-like properties. These herbs suppress prostaglandin synthesis by cox inhibition, thereby improving absorption of the chromium complexes of the invention. These herbs are relatively free from gastric ulcerogenic effects (Singh et al., Agents and Actions 18:407-412, 1986). In addition, pre-clinical acute toxicity studies have shown that salicin-containing plants do not cause hematological disturbances (American Herbal Products Association, Botanical Safety Handbook, 1997).

The compounds and herbs described above all effect gut physiology by inhibiting prostaglandin synthesis, decreasing mucus production, and lowering gastrointestinal pH. The inclusion of these compounds, as well as an enteric coating, into the oral chromium complex compositions of the invention results in a multiple component delivery system which allows delivery of these agents to the gastrointestinal tract where they work in concert to facilitate chromium absorption, thereby improving insulin sensitivity.

In a preferred embodiment, the chromium complex is coated onto microbeads. In a particularly preferred embodiment, these microbeads are sugar beadlets of various sizes, also known as nonpareils, and are commercially available from, for example, SmithKline Beecham. If the microbeads are to be used to administer the compositions of the invention to diabetic patients, the administration of other types of microbeads, such as microcrystalline cellulose, is preferred. Microcrystalline cellulose is commercially available and can be processed into beadlets of various sizes by micronization, a technique well known in the art. The microbeads are essentially a carrier for the compositions of the invention. For a description of coated beadlets, see, for example, Carstensen, J. T., Pharmaceutical Principles of solid Dosage Forms, Technonic Publishing Co., Inc., Lancaster, Pa., pp. 228-230, 1993, hereby incorporated by reference. Aqueous solutions containing the chromium complexes with or without the chelating agent components such as nicotinic acid and picolinic acid are sprayed onto the microbeads by well known methods such as suspending the microbeads in an upcurrent of air, introducing a fine spray of the active ingredients to form a coating on the outside of the microbeads, and allowing the microbeads to dry. The desired chromium complex component and alpha-lipoic acid component with or without a chelating agent may be combined into one same solution or applied using separate solutions. Optionally, the coated microbeads can be further coated with a substance to protect the active ingredients coated onto the beads, such as latex. The microbeads may be placed in a capsule prior to administration. In another preferred embodiment, the capsule or the microbeads are coated with an enteric coating to delay dissolution until reaching the small intestine.

Typically, the dosage range of chromium administered to an individual in the form of chromium picolinate, chromium nicotinate, or other chromium complex provides between about 50 and 10,000 micrograms per day of chromium; preferably between about 100 and 1,000 micrograms per day; more preferably, between about 200 and 500 micrograms per day of chromium.

Similarly, the dosage range of alpha-lipoic acid administered to an individual provides between about 10 and 1,000 milligrams per day alpha-lipoic acid; preferably between about 50 and 500 milligrams per day; and more preferably, 100 milligrams of alpha-lipoic acid are administered per day.

In some embodiments, methods of improving insulin sensitivity, reducing hyperglycemia, and reducing hypercholesterolemia with chromium complexes and alpha-lipoic acid are contemplated. The compounds of the present invention can be administered separately or as a single composition. Advantageously, a subject is administered a pharmaceutically effective dose of a chromium complex. In one embodiment, the alpha-lipoic acid is administered substantially simultaneously. In an alternative embodiment, the chromium complex is administered first and then the ALA is added second. In yet another embodiment, the ALA is administered first. If administered separately, the compounds should be given in a temporally proximate manner, e.g. within a twenty-four hour period, such that the improvement in insulin sensitivity, reduction of hyperglycemia, and/or reduction of hypercholesterolemia is enhanced. More particularly, the compounds may be given within one hour of each other. The administration can be by any of the methods of administration described above or by drug delivery methods known by one of skill in the art.

The following examples teach the methods and compositions disclosed herein for improving insulin sensitivity through the administration of at least one chromium complex in concert with alpha-lipoic acid. These examples are illustrative only and are not intended to limit the scope of the invention disclosed herein. The treatment method described below can be optimized using empirical techniques well known to those of ordinary skill in the art. Moreover, artisans of skill would be able to use the teachings described in the following examples to practice the full scope of the invention disclosed herein.

### EXAMPLE 1

### Promotion of Glucose Uptake

The synergistic effect of co-administration of at least one chromium complex and alpha-lipoic acid on the promotion of glucose uptake was investigated in vitro. Chromium picolinate and alpha-lipoic acid were incorporated into an in vitro human skeletal muscle model of Type II diabetes. The results of the co-administration of a chromium complex and alpha-lipoic acid are illustrated in Table 1. Table 1 details the synergistic effect of the co-administration of a chromium complex and alpha-lipoic acid for the promotion of glucose uptake in an in vitro human skeletal muscle model of Type II diabetes.

[image]

The combination of at least one chromium complex and ALA when studied in an in vitro human skeletal muscle model of Type II diabetes exhibited synergy with respect to its ability to promote glucose uptake. This evidences that the combination of a chromium complex and ALA, in specific ratios, can improve the impairment in insulin sensitivity that is characteristic of Type II diabetes.

### EXAMPLE 2

### Increasing Insulin Sensitivity

An adult human subject suffering from Type II diabetes is identified. The subject is orally administered a tablet containing about 200 μg chromium and 100 mg alpha-lipoic acid once per day. Over the course of several weeks, an increase in glucose sensitivity is observed. The chromium picolinate in combination with alpha-lipoic acid synergistically increase the subject's sensitivity to insulin.

It will be appreciated that although specific embodiments of the invention have been described herein for the purposes of illustration, various modifications may be made without deviating from the spirit and scope of the invention. Accordingly, the invention is not limited except as by the appended claims.

What is claimed is:

1. A composition consisting essentially of a pharmaceutically effective amount of at least one chromium complex and a pharmaceutically effective amount of alpha-lipoic acid.

2. The composition of claim 1, wherein said chromium complex is selected from the group consisting of chromium picolinate, chromium nicotinate, chromic tripicolinate, chromic polynicotinate, chromium chloride, chromium histidinate, and chromium yeasts.

3. The composition of claim 1, wherein said composition is incorporated into a pharmaceutically effective carrier.

4. The composition of claim 3, wherein said pharmaceutically effective carrier is selected from the group consisting of a tablet, capsule, microbead, emulsion, powder, granule, suspension, syrup and elixir.

5. The composition of claim 4, wherein said microbead is a sugar beadlet or microcrystalline cellulose beadlet and said at least one chromium complex and said alpha-lipoic acid are coated on said beadlet.

6. The composition of claim 4, wherein said tablet, capsule, or microbead is coated with an enteric coating.

7. The composition of claim 1, wherein said chromium complex and said alpha-lipoic acid are in a ratio of between about 1:25 to 1:1000 (w/w).

8. A composition consisting essentially of a pharmaceutically effective amount of at least one chromium complex, a pharmaceutically effective amount of alpha-lipoic acid, and a chelating agent.

9. The composition of claim 8, wherein said chelating agent is picolinic acid, nicotinic acid, or both.

10. A composition consisting essentially of a pharmaceutically effective amount of at least one chromium complex, a pharmaceutically effective amount of alpha-lipoic acid, and at least one of a cyclooxygenase inhibitor, a mucolytic, and a salicin-containing herb.

11. The composition of claim 10, wherein said cyclooxygenase inhibitor is selected from the group consisting of indomethacin, ibuprofen, acetaminophen, and naproxen.

12. The composition of claim 10, wherein said salicin-containing herb is selected from the group consisting of Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (balm of Gilead) and Salix alba (white willow).

13. The composition of claim 10, wherein said mucolytic is guaifenesin.

14. A method of improving insulin sensitivity in a subject in need thereof comprising:
identifying a subject suffering from insulin insensitivity; and
administering to said subject a composition consisting essentially of a pharmaceutically effective dose of alpha-lipoic acid and a pharmaceutically effective dose of at least one chromium complex selected from the group consisting of chromium picolinate, chromium nicotinate, chromic tripicolinate, chromic polynicotinate, chromium chloride, chromium histidinate, and chromium yeasts.

15. The method of claim 14, further comprising administering at least one uncomplexed chelating agent.

16. The method of claim 15, wherein said chelating agent is picolinic acid, nicotinic acid, or both.

17. The method of claim 14, further comprising administering at least one of a cyclooxygenase inhibitor, a mucolytic, and a salicin-containing herb.

18. The method of claim 17, wherein said at least one cyclooxygenase inhibitor is selected from the group consisting of indomethacin, ibuprofen, acetaminophen, and naproxen.

19. The method of claim 17, wherein said salicin-containing herb is selected from the group consisting of Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (balm of Gilead) and Salix alba (white willow).

20. The method of claim 17, wherein said mucolytic is guaifenesin.

21. A method of reducing hyperglycemia in a subject in need thereof comprising:
identifying a subject suffering from hyperglycemia; and
administering to said subject a composition consisting essentially of a pharmaceutically effective dose of alpha-lipoic acid and a pharmaceutically effective dose of at least one chromium complex selected from the group consisting of chromium picolinate, chromium nicotinate, chromic tripicolinate, chromic polynicotinate, chromium chloride, chromium histidinate, and chromium yeasts.

22. The method of claim 21, further comprising administering a chelating agent.

23. The method of claim 22, wherein said chelating agent is picolinic acid, nicotinic acid, or both.

24. A method of reducing hypercholesterolemia in a subject in need thereof comprising:
identifying a subject suffering from hypercholesterolemia; and
administering to said subject a composition consisting essentially of a pharmaceutically effective dose of alpha-lipoic acid and a pharmaceutically effective dose of at least one chromium complex selected from the group consisting of chromium picolinate, chromium nicotinate, chromic tripicolinate, chromic polynicotinate, chromium chloride, chromium histidinate, and chromium yeasts.

25. The method of claim 24, further comprising administering a chelating agent.

26. The method of claim 25, wherein said chelating agent is picolinic acid, nicotinic acid, or both.
